(19) United States
(12) Patent Application Publication
(10) Pub. No.: US 2024/0130679 A1
(43) Pub. Date: Apr. 25, 2024

(54) SIMPLIFIED CONNECTED ECG WATCH

(71) Applicant: Withings, ISSY LES MOULINEAUX (FR)

(72) Inventors: Marie WILLEMET, ISSY LES MOULINEAUX (FR); Walid RAFIK, ISSY LES MOULINEAUX (FR)

(21) Appl. No.: 18/480,979

(22) Filed: Oct. 3, 2023

(30) Foreign Application Priority Data
Oct. 23, 2022 (FR) 2211022

(51) Int. Cl. A61B 5/00; A61B 5/318; G04B 47/06; G04G 21/02

(52) U.S. Cl. CPC A61B 5/681; A61B 5/318; G04B 47/063; G04G 21/025

(57) ABSTRACT

A portable electronic device is proposed, configured for positioning on a user's wrist, for performing an electrocardiogram. The device includes exactly two ECG electrodes, the potential being imposed on one of the two ECG electrodes. The two electrodes consist of a first ECG electrode at the case back and configured to be in contact with the skin of the wrist, and a second ECG electrode.

### CROSS-REFERENCE TO RELATED APPLICATIONS

This application claims priority to French Patent Application No. FR2211022, filed Oct. 24, 2022, the entire content of which is incorporated herein by reference in its entirety.

### FIELD

The present invention relates to the field of connected watches and in particular to connected watches capable of performing an electrocardiogram (ECG or EKG). By watch is meant a wearable device, the preferred position of which is the wrist. The present invention also concerns the field of so-called hybrid connected watches, i.e. connected watches with a visual appearance closer to conventional mechanical watches, thanks in particular to the presence of a gear train and mechanical hands to indicate at least the time (hour hand and minute hand).

### BACKGROUND

An ECG is a measurement of the heart's electrical activity. An electrical impulse passes through the heart with each contraction, and the ECG is a tracing obtained by recording electrical potentials. The ECG is one of the key measurements for cardiovascular monitoring. Thanks to its integration into a watch, any user may perform an ECG on a regular basis, enabling better analysis and prevention of cardiac risks. To perform an ECG, several electrodes are positioned on the human body, in order to detect different components of the electrical signals, known as leads. In one of its simplest forms, the ECG provides a bipolar measurement between the right and left arms, known as lead I or DI.

The test is painless, passive and non-invasive (no current is injected into the skin), and may be performed in less than a minute. Although the theoretical concept of integrating an ECG into a consumer device has been presented for several years (see U.S. Pat. No. 5,289,824 in particular), concrete, effective implementation has only recently been achieved. This is a major technological innovation.

A Review of Methods for Non-Invasive Heart Rate Measurement on Wrist” (de Pinho Ferreira & Al, 2020) describes the state of the art in wrist cardiac measurement. This paper explains that unipolar ECG measurement from a single point on the human body would not be possible due to its differential nature, as well as the common mode of this signal, which is buoyant and may vary considerably. To obtain the highest Signal to Noise Ratio (SNR), two electrodes are usually placed on either side of the heart, where cardiac action potentials occur. Unfortunately, the signal is rapidly attenuated when the electrodes are moved away from the heart, and particularly if both electrodes are placed on the same side of the heart, such as along a single arm. However, if the measurement is taken from both hands, the amplitude remains high enough to extract the heart rate conveniently. Thus, the conventional approach is to place a device on the wrist to be touched by the other hand, thus ensuring electrical contact with the skin of both arms, as shown in FIG. 11 illustrating the state of the art: two electrodes (ECG_N and VCM) are placed under the device, in contact with the wrist of one arm (e.g. the left arm), and a top electrode (ECG_P) is used to place a finger on the opposite hand (e.g. that of the right arm).

ECG watches (hereinafter referred to as “ECG watches”) are few and far between on the market in 2022. These include the Withings Move ECG, Withings ScanWatch, Apple Watch and Samsung Galaxy.

As described above, these watches all use three ECG electrodes, in a configuration similar to that of FIG. 11 with regard to the contacts between the arms and the electrodes. In particular, lead I ECG measurement involves measuring the difference in electrical potential between the left and right upper limbs. The three electrodes therefore comprise a negative electrode (ECG_N) in contact with one arm, a neutral electrode in contact with the same arm and a positive electrode (ECG_P) in contact with the other arm. This arrangement makes movement simple and repeatable, by placing a single electrode in contact with the arm to be moved. The role of the neutral electrode is to fix the body at a common mode (a mean potential). This allows the signal to be centered around a mean potential, and actively eliminates the noise commonly observed on both limbs, mainly electromagnetic interference due to electrical power sources (noise at 50 or 60 Hz).

Implementing an ECG measurement device in a watch presents a number of technical difficulties, not least because the electrical signals to be identified are of low amplitude and their acquisition is frequently noisy. Correct positioning of the electrodes and management of the electrical chain between the electrodes and the electronic module that manages the ECG are crucial.

### SUMMARY

An aspect of the invention is directed to simplifying the architecture of such an ECG-watch, in particular to reduce noise sources, to limit the number of mechanical parts and/or manufacturing steps, and to simplify electronic assembly.

To this end, the present description relates to a portable electronic device configured to be positioned on a user's wrist, the portable device enabling an electrocardiogram, ECG, to be performed, the device comprising: a case back, configured to be at least partially in contact with the skin of the wrist, the device further comprising exactly two ECG electrodes, the two ECG electrodes consisting of: a first ECG electrode, made of conductive material, at the case back and configured to be in contact with the skin of the wrist, a second ECG electrode, made of conductive material. To perform an electrocardiogram, the device further comprises an ECG electronic module, electrically connected to the first ECG electrode and to the second ECG electrode, and configured to impose a potential on one of the two ECG electrodes and to measure a potential of the other of the two ECG electrodes. The second ECG electrode is arranged on the portable electronic device so that it may be brought into contact with the user's arm opposite that which is in contact with the first ECG electrode.

The portable electronic device directly measures the potential difference between one of the two ECG electrodes, e.g. the one in contact with the wrist, and an electrical potential imposed on the other electrode, e.g. the one incorporated in the watch bezel. The portable electronic device measures the potential difference between the variable potential at one of the two ECG electrodes, and an imposed potential at the other electrode, unlike a three-electrode configuration where the potential difference is calculated between two ECG electrodes with variable potential. The imposed potential is typically constant, to simplify the electronic architecture.

This configuration of two ECG electrodes in a watch has not been proposed before, due to the need to limit electromagnetic interference from electrical power sources. However, modern watches operate on a reduced power supply from a battery inside the watch. Continuous connection to a power supply network is no longer necessary. The inventors had the idea of proposing a two-electrode architecture, despite the difficulties that such a solution might suggest. Indeed, apart from the question of interference, the arrangement of the two electrodes is necessarily different from the conventional architecture of watches with three ECG electrodes. The inventors therefore developed an ECG watch architecture with just two ECG electrodes, and carried out test campaigns. They found that, as long as the measurement was carried out without direct contact with a mains-connected power source, electromagnetic interference was minimal and did not require the presence of the neutral electrode. The inventors then proposed this new, simplified two-electrode ECG architecture for ECG measurement on a watch.

This architecture offers numerous benefits. It simplifies the architecture of the parts, since only one electrode is required at the case back, as opposed to two previously. This electrode is consequently optimized, as its surface area may be enlarged. In addition, an aspect of the invention makes it possible to use fewer components in the electronic circuit, while avoiding amplitude loss due to the injection of a common-mode potential via the imposed-potential electrode.

In an embodiment, the device further comprises an enclosure, a crystal and a bezel mounted on the enclosure and surrounding the crystal, wherein the bezel comprises a bezel body and the bezel body comprises the second ECG electrode.

In an embodiment, the bezel may be rotated relative to the enclosure.

In an embodiment, the case comprises a side wall, the side wall comprising an opening into which a rotatable and/or translatable crown is inserted, the second ECG electrode being formed by the crown.

In an embodiment, the imposed potential is a constant potential. This simplifies the electronic architecture and the processing of electrical signals to generate an ECG.

In an embodiment, the ECG module is configured to impose the potential on the second ECG electrode.

In an embodiment, the ECG module is configured to impose the potential on the negative electrode.

In an embodiment, the ECG module comprises an analog front end adapted to receive a maximum voltage amplitude, the ECG module being configured to impose the constant potential at a value approximately equal to half the maximum voltage amplitude.

In an embodiment, the ECG module is configured to impose the potential at a value between 0.5V and 2V, in particular between 0.8V and 1.0V. In particular, the imposed potential is constant.

In an embodiment, the case back comprises an optical sensor, with the first electrode at least partially surrounding the optical sensor.

In an embodiment, the first electrode surrounds the optical sensor to an angular extent greater than 180°.

In an embodiment, the first electrode completely surrounds the optical sensor.

In an embodiment, the first electrode is ring-shaped.

In an embodiment, the first electrode is in the form of a metal body.

In an embodiment, the first electrode is in the form of a metal coating.

In an embodiment, the portable electronic device is a watch, the watch being, for example, a hybrid watch with mechanical hands.

The present invention also relates to a method of taking an electrocardiogram, ECG, using a device as defined above, during which the user places one arm in contact with the first ECG electrode and another arm in contact with the second ECG electrode.

The present invention also relates to the use of a device as previously described to perform an electrocardiogram.

### BRIEF DESCRIPTION OF THE DRAWINGS

Further features, details and advantages will become apparent from the detailed description below, and from an analysis of the appended drawings, on which:

FIG. 1 This figure shows a top view (along a Z direction) of the ECG watch, where the dial, crystal and bezel in particular are visible, according to a first embodiment (the hands are not shown).

FIG. 2. This figure shows a view from below (along a Z direction) of the ECG watch, where the case back and the enclosure in particular are visible, according to the first embodiment.

FIG. 3 This figure shows a cross-sectional view along the XZ plane of an ECG watch, according to one embodiment.

FIG. 4 This figure shows a diagram of the ECG-watch with some of its components, including electronics.

FIG. 5 This figure illustrates the operation of the ECG-watch.

FIG. 6 This figure shows a top view (along a Z direction) of the ECG-watch in a second embodiment.

FIG. 7 This figure shows a perspective view of the underside of the ECG watch in the second embodiment.

FIG. 8 This figure shows a perspective bottom view of ECG watches in other embodiments.

FIG. 9 FIG. 10 These figures show comparative test results of the ECG-watch according to the second embodiment with an ECG-watch with 3 ECG electrodes configuration.

FIG. 11 This figure shows an ECG watch according to the state of the art.

### DETAILED DESCRIPTION

The present description relates to a portable electronic device comprising an electrocardiogram sensor (hereinafter: ECG sensor). In a particular embodiment, which is the one illustrated, the portable electronic device is a watch (hereinafter: ECG-watch). The ECG-watch may comprise a wristband. However, for the purposes of this description, the term ECG-watch does not necessarily include the strap, which is usually manufactured elsewhere and may be assembled at points of sale.

The portable electronic device is connected, so that it may exchange data remotely (wirelessly) in a bidirectional way with a terminal, such as a smartphone. The connection may be via BLUETOOTH® (a short-range wireless technology standard), such as BLUETOOTH® Low Energy (BLE). In particular, the data exchanged from the ECG-watch to the terminal is ECG data acquired by the ECG-watch. The ECG-watch may also receive data from the terminal (time, alarm data, notifications, etc.).

In an embodiment, the ECG-watch is a hybrid watch, i.e. a watch with a dial and hands to indicate the hours and minutes.

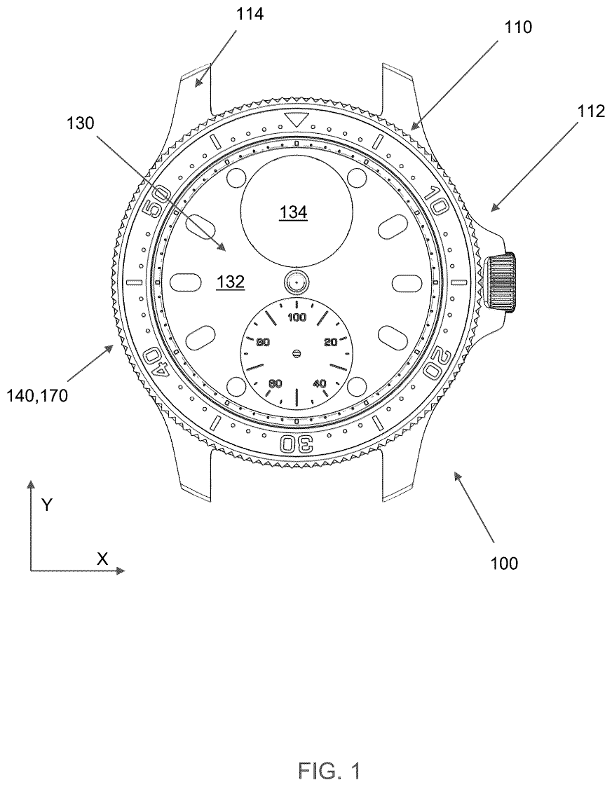

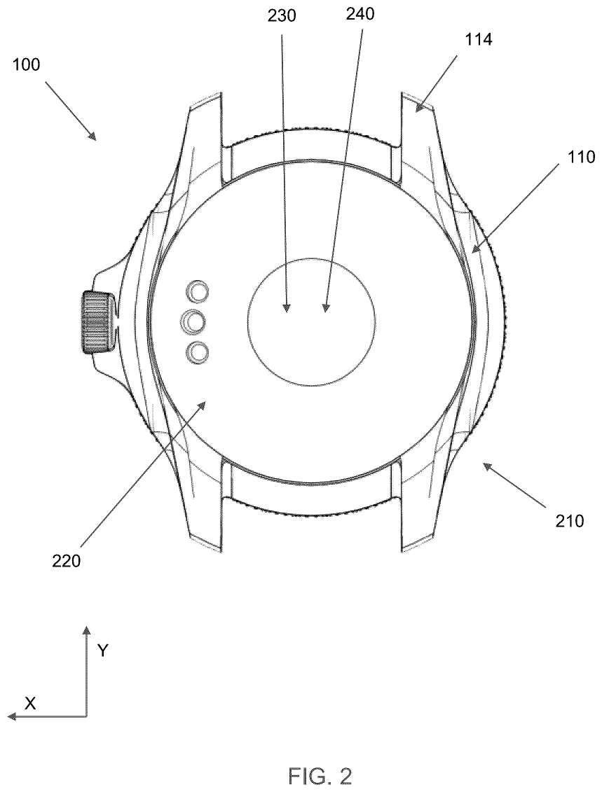

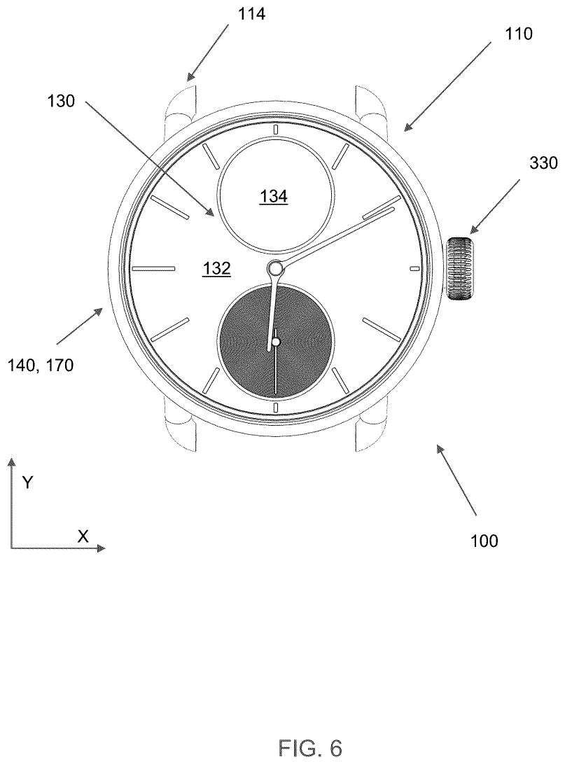

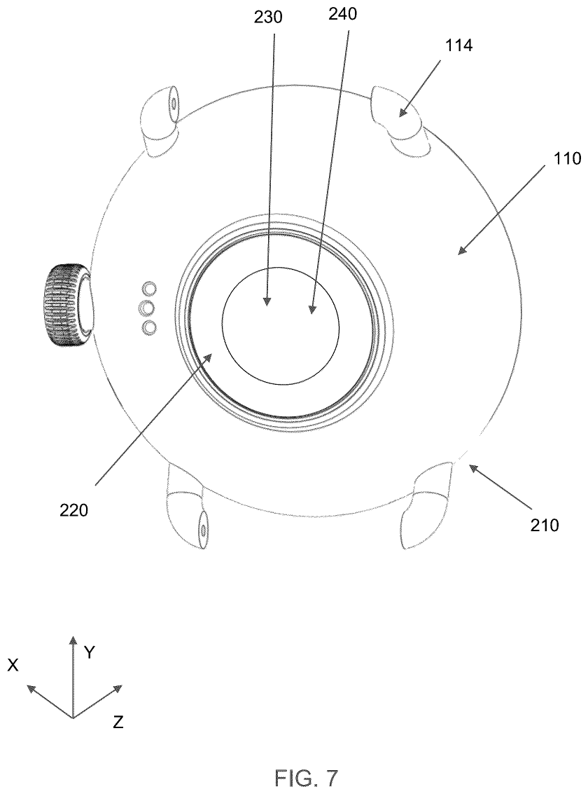

FIGS. 1 to 3 show an electronic ECG 100 watch of the hybrid type (with a dial, mechanical hands and possibly a display integrated into the dial) according to a first example, and FIGS. 6 to 7 show an electronic ECG 100 watch according to a second example.

In the present description, the notion of “top” and “bottom” is defined in relation to the Z direction, the top being in the direction of the crystal and the bottom being in the direction of the case back, which will be described below.

The ECG 100 watch may comprise an enclosure 110 and a case back 210 which is configured to be at least partially in contact with the skin of the user's wrist. The enclosure 110 and case back 210 are integral with each other. In an embodiment, as shown in the figures, the enclosure 110 and case back 210 are two separate parts. In an embodiment not shown in the figures, the case back 210 is an integral part of the middle 110. The middle 110 may comprise a side wall 112, which is generally visible when the ECG 100 watch is worn on the wrist. The middle 110 may include lugs 114 (two pairs, on either side of the middle 110) for attaching a bracelet (not shown in the figures). The middle 110 may include a plurality of parts.

The ECG 100 watch may also include a crystal 130, usually mounted on the enclosure 110, so that the glass 130 is fixed. The crystal 130 is or may comprise a typically transparent protective glass and may be made of glass, ceramic, plastic or any transparent material. The outline of the crystal 130 is typically circular.

In the case of a hybrid watch, below the crystal 130, the ECG 100 watch also comprises a dial 132 with hands (physical hands, not shown in the figures). The dial 132 may further accommodate a display 134 (for example with an opening in the dial that allows a display positioned just below the dial to be made visible), which for example occupies a small space below or in the dial 132. The cover 130 protects these parts and allows them to be seen through.

In the case of an Apple Watch-type smartwatch, below the crystal 130, the ECG-watch comprises a screen, not shown here, which occupies a width close to the width of the ECG-watch 100. In an embodiment, the screen may display hands. The crystal 130 is then the protective glass of the display.

The ECG 100 watch may also include a bezel 140, mounted on the enclosure 110. The bezel 140 is positioned around the crystal 130 (radially external to the crystal). Bezel 140 is a ring-shaped part, essentially revolving around the Z direction (with a few modifications).

In the example shown in FIGS. 1 to 3, the bezel 140 is rotatably mounted relative to the enclosure 110. Rotation may take place step by step (for example, notch by notch). In this way, the user may turn the bezel 140. Alternatively, in the example shown in FIGS. 6 and 7, bezel 140 is fixed relative to enclosure 110. In this variant, the bezel 140 is not rotatable relative to the enclosure 110.

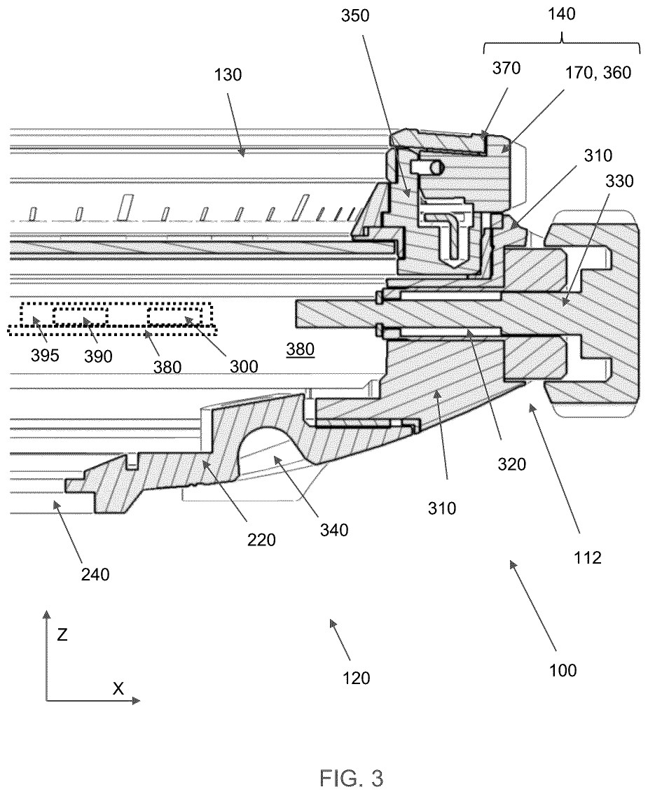

FIG. 3 shows a cross-sectional view of the ECG-watch 100 according to the first example. In particular, the enclosure 110 comprises a main body 310 (or enclosure body), which includes the side wall 112. In FIG. 3, the main body 310 comprises an opening 320 at the level of the side wall 112 to allow the crown 330 to be fitted. In particular, the crown 330 acts as a user interface between the ECG 100 watch and the user. To this end, the crown 330 may be rotated (here around the X axis) and/or translated (here along the X axis).

The case back 210 may be mounted on the main body 310. In the cross-sectional view of FIG. 3, the case back 210 comprises a recess 340, which is a positioning stud for a battery charging station. The enclosure 110 may also include a bezel support 350, mounted on the main body 310. In particular, the bezel 140 is mounted on the bezel support 350, in a radially external position. The crystal 130 is also mounted on the bezel support 350, in a radially internal position.

The bezel 140 may comprise a bezel body 360, which is mounted on the enclosure 110. The bezel body 360 may directly incorporate (by engraving or otherwise) the traditional watch decoration, so that the bezel 140 is formed integrally by the bezel body 360. Alternatively, as shown in FIG. 3, the bezel 140 further comprises a display ring 370 (also known as a “decor”) which may be mounted (e.g. by gluing, e.g. with double-sided adhesive) on an upper face of the bezel body 360. The decor may then include visual markers.

The 360 bezel body may be made of conductive material, such as metal (e.g. stainless steel or titanium alloy). Alternatively, the 360 bezel body may be made of filled plastic or conductive ceramics. Alternatively, the 360 bezel body may be non-conductive and a conductive coating is applied to all or part of the 360 bezel body (e.g. from the top face or outer side face to the notches on the bottom face).

The ECG 100 watch includes an optical sensor 230, shown in FIG. 2. The optical sensor 230 comprises at least one light source (e.g. LED), for emitting light, and at least one photodiode, for receiving light. The optical sensor 230 is typically a PPG (photoplethysmography) sensor. The optical sensor 230 may be positioned behind a lens 240, for example made of glass, which interfaces with the skin of the wrist. Document PCT/EP2021/058955, in the name of Withings and which is incorporated herein in its entirety by reference, describes the optical sensor in detail, which may be found on the Withings ScanWatch.

To recover the electrical signals generated by the human body, the ECG-watch 100 includes an ECG sensor. In particular, the ECG sensor comprises a set of electrodes (referred to as ECG electrodes and described in detail below) and an ECG electronic module 300 (illustrated schematically in FIG. 3), to which the ECG electrodes are electrically connected.

By electrode we mean a conductive part capable of receiving or transmitting an electric current, an electric potential or an electric voltage. The part may be made of conductive material or include a conductive coating. By “conductor” we mean “conductor of electricity”.

According to the present description, the ECG-watch 100 comprises exactly two ECG electrodes for performing an ECG, including a first ECG electrode and a second ECG electrode. Thus, unlike conventional ECG-watches which require three ECG electrodes in order to perform an ECG, the present description proposes to simplify the architecture of the ECG-watch 100, as will be explained in more detail later.

As shown in FIGS. 2 and 3, the first ECG electrode 220 is located at the case back 210 so as to be in contact with the skin of the wearer's wrist on which the ECG-watch 110 is worn. The first electrode 220 is electrically connected to the ECG module 300. The first ECG electrode 220 may at least partially surround the optical sensor 230.

The first electrode 220 is made of a conductive material, such as metal (e.g. stainless steel or titanium alloy). In the example shown in FIGS. 1 to 3, the first electrode 220 is in the form of a metal body. By metal body we mean a part whose maximum thickness is greater than 0.1 mm.

In the variant illustrated in FIGS. 6 and 7, the first electrode 220 may be formed by a conductive coating deposited on a surface (which is itself conductive or non-conductive). In particular, the first electrode 220 takes the form of a metal coating on the lens 240 where the optical sensor 230 is located.

In the two examples shown in FIGS. 1 to 7, the first electrode 220 has an annular shape.

In the examples shown, the first electrode 220 at least partially surrounds the optical sensor 230. In particular, the first electrode 220 may completely surround the optical sensor 230, as shown in FIGS. 2 and 7.

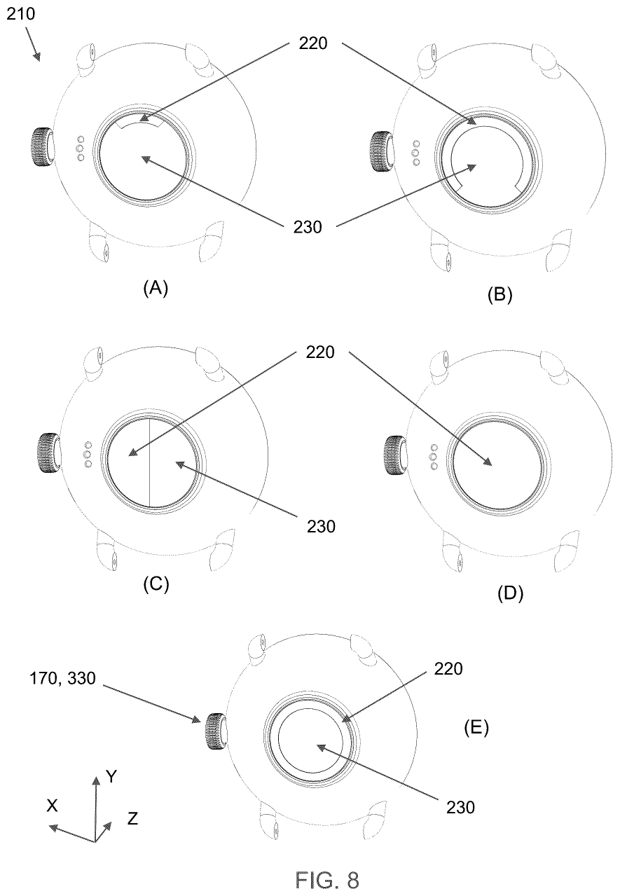

FIG. 8 shows further examples of arrangements for the first electrode 220.

In example (A) in FIG. 8, the first electrode 220 may extend around the optical sensor 230 over an angular range of less than 180°, for example 90°. The first electrode 220 is shown at the top in the Y direction, but may alternatively be located anywhere around the optical sensor 230.

In example (B) in FIG. 8, the first electrode 220 may extend around the optical sensor 230 over an angular range greater than 180°, but less than 360°, for example around 270°. The first electrode 220 is shown at the top in the Y direction, but may alternatively be located anywhere around the optical sensor 230.

In the example (C) shown in FIG. 8, the first electrode 220 and the optical sensor 230 may be formed by two half-discs facing each other. The first electrode 220 is shown on the left in the X direction, but may alternatively be on the right in the X direction.

In the example (D) shown in FIG. 8, the ECG 100 watch does not include an optical sensor 230, and the first electrode 220 is in the form of a disc arranged substantially in the center of the case back 210.

The second electrode 170 is also electrically connected to the ECG module 300.

As it can be seen in FIGS. 1 and 3, the second ECG electrode 170 may be located on the bezel 140 so that it may easily be in contact with the skin of the user's other hand. As illustrated in the figures, the second electrode 170 is the bezel body 360, i.e. the entire bezel 140 or part of it (accessible by the user) forms the electrode 170. The user may thus touch the bezel at any point. Compared with a crown as electrode, the ECG-watch 100 may be worn on either the left or right hand, without the gestures required to perform an ECG being the same for a user wearing the watch on the left or right (symmetry of use), unlike the electrode crowns described in the introduction.

Alternatively, as shown in example (E) in FIG. 8, the second ECG electrode 170 is formed by the crown 330. To take an ECG measurement, the user places his free hand in contact with the crown 330.

Alternatively, in a non-illustrated example, the second electrode is located on or forms part of a button in the enclosure.

The second electrode 170 is made of conductive material, such as metal (e.g. stainless steel or titanium alloy). In the examples shown, the second electrode 170 is in the form of a metal body.

The enclosure 110 (and in particular the enclosure back 210, the dial 132 and the main body 310 of the enclosure 110 ) defines an internal volume 380 suitable for accommodating various components, such as electronic components. These electronic components are thus protected from water or dust (with the appropriate seals, notably provided by the aforementioned gaskets).

The ECG 300 module is shown schematically dotted in FIG. 3 and positioned schematically. The two ECG electrodes 220, 170 are electrically connected to the ECG module 300. The ECG 300 module is configured to retrieve electrical signals from the human body and, after processing, generate an electrocardiogram. The ECG 300 module may be mounted on an electronic board 380 (shown schematically as a dotted line in FIG. 3 and positioned schematically), of the printed circuit board (PCB) type, where other components of the ECG-watch are also mounted.

The ECG 300 module includes an analog front-end (AFE). The analog front-end is configured to receive at two inputs (a positive and a negative input) the signals from the two ECG electrodes 220, 170 respectively (which are thus defined as a positive and a negative electrode) and to output an ECG signal. The analog front-end is, for example, the AD8233 from Analog Devices. The analog front-end accepts a maximum voltage amplitude, corresponding to the analog front-end supply voltage. Typically, this maximum amplitude may be between 1V and 4V, for example around 1.8V.

The ECG module 300 is configured to impose a potential on one of the two ECG electrodes. In particular, the potential is imposed passively by the analog front end. Beneficially, the imposed potential is a constant potential. In an embodiment, the constant potential is equal to approximately half the maximum voltage amplitude of the analog front-end. Approximately means plus or minus 10% of the value. In particular, the imposed potential is between 0.5V and 2V, for example between 0.8V and 1.0V, especially 0.9V.

Indeed, if a potential were not imposed on one of the two ECG electrodes, the two ECG electrodes would be left “floating”, i.e. they would not be connected to a reference value. There would be an amplitude potential difference between the 2 electrodes, but the individual potentials could take on a multitude of values. However, as explained above, the analog front-end accepts a limited voltage amplitude. In the example in which this maximum amplitude is around 1.8V, imposing a constant potential of around 0.9V on one of the two electrodes brings the measurement within the acceptable amplitude for the ECG 300 module. The measurement will then oscillate around 0.9V, and will be measurable by the analog front end. In this way, the inventors have determined that, by positioning themselves at half maximum amplitude, the acquisition of deviations is optimized upwards or downwards.

Beneficially, the ECG module 300 imposes the potential on the second ECG electrode 170, which is configured to be in contact with the arm opposite the one carrying the ECG device 100. Thus, unlike a conventional 3-electrode ECG configuration where the reference electrode is placed at the case back, the ECG 300 module imposes the potential on the ECG electrode in contact with the free hand (i.e. the hand linked to the wrist where the watch is not located). In particular, the ECG module 300 is configured to impose the potential on the negative electrode, which is notably the second ECG electrode 170. The inventors realized that imposing the potential on the electrode in contact with the free hand makes it possible to reduce potential fluctuations induced by contact of this free hand with the bezel and to reduce micro-variations induced by the nervous system (unlike contact between the first electrode 220 at the case back and the arm on which the ECG 100 device is mounted, notably because the arm remains immobile and inactive during measurement).

The potential at the other electrode is left free by the ECG module 300, so that the potential of this electrode corresponds to the potential of the user's body (when there is contact) and varies according to the user's heartbeat. Beneficially, the ECG 300 module leaves the potential of the first electrode 220 free.

The optical sensor 230 is connected to a PPG 390 module, also positioned in the internal volume, which may also be mounted on the electronic board 380 of the ECG 100 watch. The PPG 390 module is configured to generate the setpoints for the light sources of the optical sensor 230 and to recover the electrical signals from the photodiodes.

A control unit 395 controls the on-board electronics of the ECG-watch 100. The control unit 395 may, for example, include or partially include the ECG module and the PPG module.

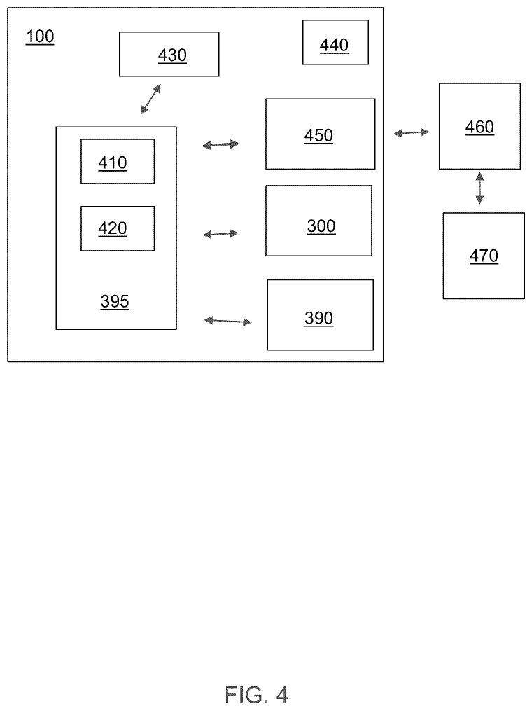

As shown in FIG. 4, which schematically illustrates the device 100, the control unit 395 may comprise one or more processor(s) 410 and a memory 420 which stores instructions suitable for execution by the processor 2302.

The ECG 100 watch may also include an accelerometer 430, connected to the control unit 395 (for monitoring sleep, activity, etc.).

To supply the various components with electrical energy, the ECG-watch 100 includes a battery 440, for example a rechargeable battery. The battery 440 is configured to power the ECG module 300 in particular. The recess 340 described above enables the case back 210 to be placed on a charging station to recharge the battery 440.

The ECG-watch 100 includes a wireless communication module 450, such as a BLUETOOTH® or BLUETOOTH® Low Energy module or a Wi-Fi module or a cellular module (GSM, 2G, 3G, 4G, 5G, Sigfox, etc.), which enables it to communicate bidirectionally with at least one external terminal 460, such as a cell phone. The external terminal 460 may then communicate (bidirectionally) with a remote server 470 for data storage and processing. Alternatively or additionally, the wireless communication module 440 may communicate directly with the remote server 470, for example via the cellular network or via a Wi-Fi network. Data obtained by the ECG-watch 100, such as an electrocardiogram, but also indications of heart rate, activity or oxygen saturation, are transmitted to the external terminal 460 via the wireless communication module 440. The control unit 395 may process certain signals before sending them, to limit data size.

The PPG module 390 and the ECG module 300 are also connected to the control unit 395 or are integrated and/or partially integrated into it. The ECG 300 module is known per se and will not be described in detail. Various types of electronic components may be included in the ECG 300 module (processor, resistor, capacitor, etc.).

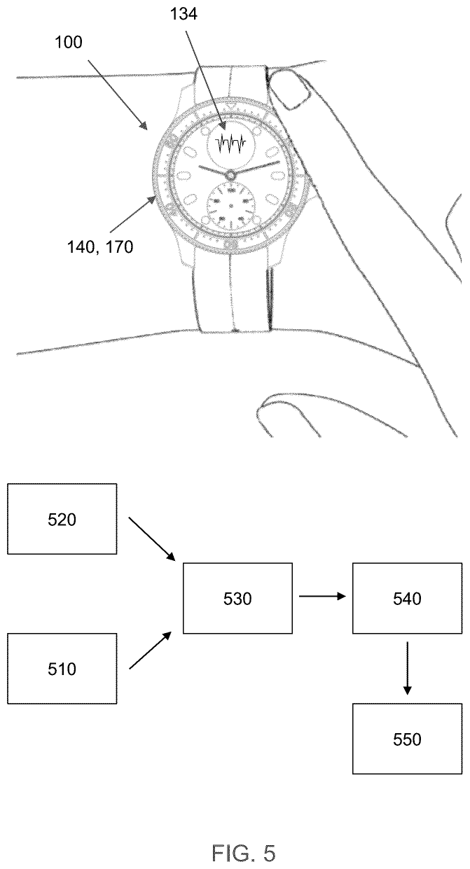

The operation of the ECG 100 will be explained below, with reference to FIG. 5.

The ECG-watch 100 is initially placed on a user's wrist. The user then selects the ECG measurement program, for example via crown 330 and display 134.

Then, as shown in FIG. 5, the user places at least one finger, or the palm of his or her hand, on the second ECG electrode 170 formed, in the example shown, by the bezel 140. ECG measurement is thus very simple for the user, who simply places his hand on the bezel 140.

A potential is imposed on one of the two ECG electrodes in step 510. Beneficially, the potential is imposed by the ECG module 300 on the second ECG electrode 170, which is the ECG electrode in contact with the free hand (i.e. the hand linked to the wrist where the watch is not located, i.e. the hand that may potentially move during measurement and disturb the signals). By imposing the potential on this hand, ECG quality is improved.

The potential at the other electrode is left free by the ECG module 300, so that the potential of this electrode corresponds to the potential of the user's body (when in contact) and varies according to the user's heartbeat. In a step 520, the potential of the first electrode 220 in contact with the user's wrist is measured. This wrist is generally very immobile and not subject to muscular stress during ECG measurement. As a result, the signals obtained by the first electrode 220 may be of better quality.

Steps 510 and 520 may be performed in parallel.

Then, in step 530, the ECG module 300 measures the potential difference between the first electrode 220 in contact with the wrist and the electrical potential imposed on the second electrode 170 incorporated in the bezel 140.

Then, in a manner known per se, the potential difference is amplified, filtered, digitized and analyzed by the ECG module 300 in a step 540.

Finally, in step 550, the result is displayed on screen 134, for example. Alternatively or additionally, the result is sent to the external terminal 460 and/or to the remote server 470. A notification may then be sent to the user, on screen 134 for example or on external terminal 460.

### Comparative Tests

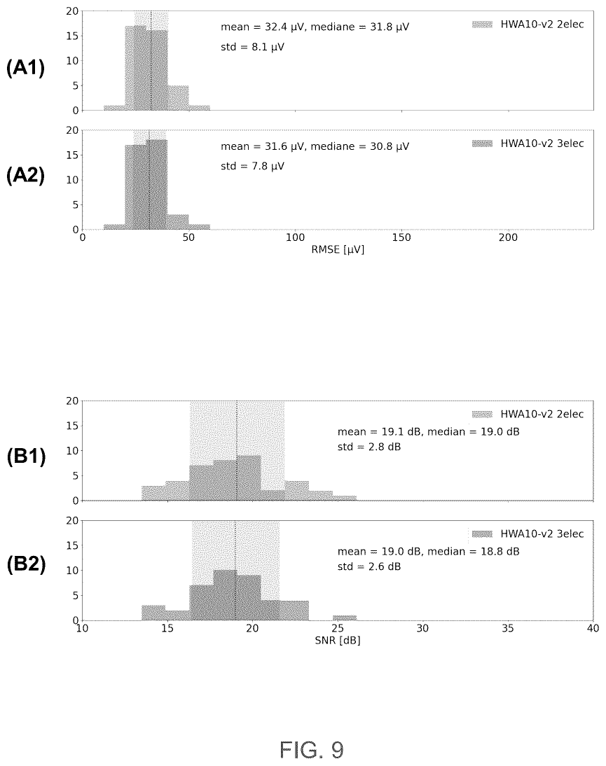

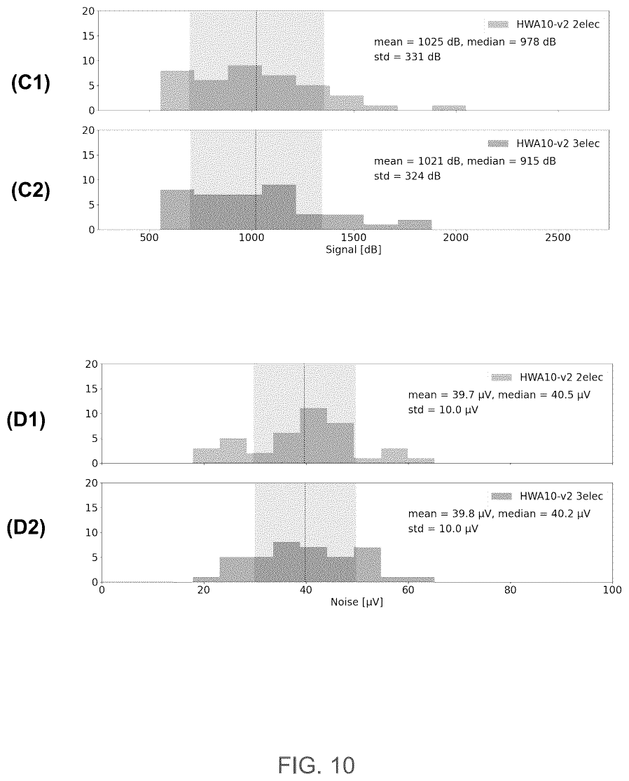

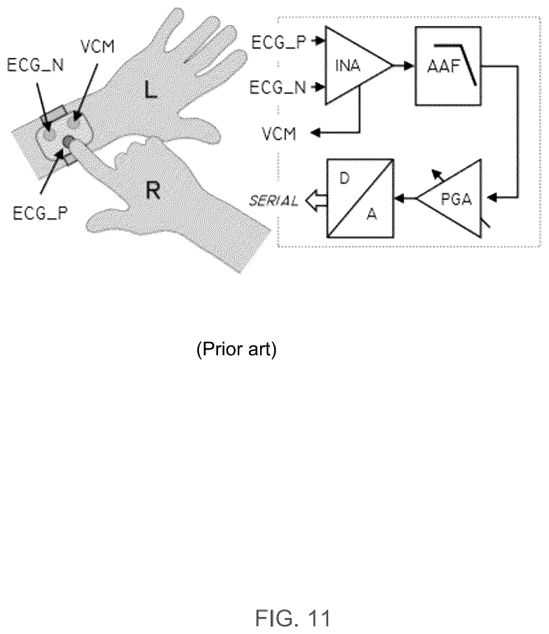

FIGS. 9 and 10 illustrate performance results for the ECG 100 watch shown in FIGS. 6 and 7. However, the person skilled in the art will understand that these results apply in a similar way to the ECG 100 watch shown in FIGS. 1 to 3.

The ECG signals recorded by the ECG 100 watch according to an embodiment of the invention with the new ECG 2-electrode configuration are compared with the ECG signals measured by a watch with a conventional 3-electrode configuration. The prototype watches used have identical mechanics, architecture and electronic components. Only the ECG electrode configuration varies. What's more, each measurement on a prototype watch was carried out simultaneously with a reference ECG measurement using a reference device (Schiller).

In the graph (A1) in FIG. 9, the ordinate represents the number of ECG recordings (40 in total) made with a watch, and the abscissa represents the root mean squared error (RMSE) of an ECG-watch 100 according to the second embodiment in relation to a reference electrocardiogram (Schiller Cardiovit FT-1). “Mean” means average and “std” means standard deviation.

The RMSE is calculated in relation to the Schiller reference signal:

$$RMSE = \sqrt{\frac{{\sum }_{i = 0}^{N} {e}_{i}^{2}}{N}}$$

with N the number of points in the signal and ei the error at point i, corresponding to the difference between the Schiller reference signal and the watch signal tested at point i.

In the graph (A2) in FIG. 9, the ordinate represents the number of ECG recordings (40 in total) made with a watch, and the abscissa represents the root mean squared error (RMSE) of a watch with a 3-electrode configuration compared with a reference electrocardiogram (Schiller Cardiovit FT-1).

In the graph (B1) of FIG. 9, the ordinate represents the number of ECG recordings (40 in total) made with a watch, and the abscissa represents the Signal to Noise Ratio (SNR) of an ECG-watch 100 according to the second embodiment, compared with a reference electrocardiogram (Schiller Cardiovit FT-1).

The signal-to-noise ratio quantifies the level of noise in the ECG signal. It is expressed in decibels (dB) and is calculated as follows: SNR=10*log(S/N), with S the signal power (S=s²/8) and N the noise power (N=n).²

In this metric, “signal s” is calculated as the amplitude of the (SQRS complex (over a 100 ms window), and “noise n” as the standard deviation of the assumed flat part of the ECG signal (a 200 ms window, which starts 250 ms before each detected QRS complex).

In the graph (B2) in FIG. 9, the ordinate represents the number of ECG recordings (40 in total) made with a watch, and the abscissa represents the Signal to Noise Ratio (SNR) of a watch with a 3-electrode configuration compared with a reference electrocardiogram (Schiller Cardiovit FT-1).

In the graph (C1) of FIG. 10, the ordinate represents the number of ECG recordings (40 in total) made with a watch and the abscissa represents the amplitude of the ECG signal of an ECG-watch 100 according to the second embodiment in relation to a reference electrocardiogram (Schiller Cardiovit FT-1).

In the graph (C2) in FIG. 10, the ordinate represents the number of ECG recordings (40 in total) made with one watch, and the abscissa represents the amplitude of the ECG signal from a watch with a 3-electrode configuration compared with a reference electrocardiogram (Schiller Cardiovit FT-1).

In the graph (D1) in FIG. 10, the ordinate represents the number of ECG recordings (40 in total) made with a watch, and the abscissa represents the noise amplitude of an ECG-watch 100 according to the second embodiment in relation to a reference electrocardiogram (Schiller Cardiovit FT-1).

On the graph (D2) in FIG. 10, the ordinate represents the number of ECG recordings (40 in total) made with a watch, and the abscissa represents the noise amplitude of a watch with a 3-electrode configuration compared with a reference electrocardiogram (Schiller Cardiovit FT-1).

With reference to all the metrics calculated and shown in FIGS. 9 and 10, it may be seen that the results obtained with the ECG 100 watch are no worse than those obtained with a watch with a 3-electrode configuration. One such watch with 3 ECG electrodes is the ScanWatch 38 mm, which, in 2019, obtained CE certification for ECG measurement.

It will be appreciated that the various embodiments and aspects of the inventions described previously are combinable according to any technically permissible combinations.

The articles “a” and “an” may be employed in connection with various elements and

components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

1. A portable electronic device configured to be positioned on a user's wrist, the portable device enabling an electrocardiogram, ECG, to be performed, said device comprising:
a case back, configured to be at least partially in contact with the skin of the wrist,
exactly two ECG electrodes, the two ECG electrodes consisting of:
a first ECG electrode, made of conductive material, at the case back and configured to be in contact with the skin of the wrist,
a second ECG electrode, made of conductive material,
an ECG electronics module, electrically connected to the first ECG electrode and the second ECG electrode, and configured to impose a potential on one of the two ECG electrodes and to measure a potential of the other of the two ECG electrodes, in order to perform an electrocardiogram.

2. The device of claim 1, further comprising an enclosure, a crystal and a bezel mounted on the enclosure and surrounding the crystal, wherein the bezel comprises a bezel body and the bezel body comprises the second ECG electrode.

3. The device as claimed in claim 1, wherein the enclosure comprises a side wall, the side wall comprising an opening into which is inserted a crown which is rotatable, the second ECG electrode being formed by the crown.

4. The device as claimed in claim 3, wherein the crown is rotatable and translatable.

5. The device according to claim 1, wherein the imposed potential is a constant potential.

6. The device according to claim 1, wherein the ECG electronics module is configured to impose the potential on the second ECG electrode.

7. The device according to claim 1, wherein the ECG electronics module comprises an analog front end adapted to receive a maximum voltage amplitude, the ECG module being configured to impose a constant potential at a value approximately equal to half the maximum voltage amplitude.

8. The device according to claim 7, wherein the ECG electronics module is configured to impose a potential at a value between 0.5V and 2V.

9. The device according to claim 7, wherein the ECG electronics module is configured to impose a potential at a value between 0.8V and 1.0V.

10. The device according to claim 1, wherein the case back comprises an optical sensor, the first ECG electrode at least partially surrounding the optical sensor.

11. The device according to claim 10, wherein the first ECG electrode surrounds the optical sensor to an angular extent greater than 180°.

12. The device according to claim 10, wherein the first ECG electrode completely surrounds the optical sensor.

13. The device according to claim 1, wherein the first ECG electrode has an annular shape.

14. The device according to claim 1, wherein the first ECG electrode is in the form of a metal body.

15. The device according to claim 1, wherein the first ECG electrode is in the form of a metal coating.

16. The device according to claim 1, wherein the portable electronic device is a watch.

17. The device according to claim 16, wherein the watch is a hybrid watch with mechanical hands.

18. A method of taking an electrocardiogram, ECG, using a device according to claim 1, the method comprising contacting the first ECG electrode with a user's arm and contacting the second ECG electrode with the user's other arm.
